(19) United States
(12) Patent Application Publication
(10) Pub. No.: US 2005/0148895 A1
(43) Pub. Date: Jul. 7, 2005

(54) Method and apparatus for ECG derived sleep monitoring of a user

(76) Inventors: Dale Julian Misczynski, Austin, TX (US); Vladislav Bukhman, East Northport, NY (US)

Correspondence Address: Dale J. Misczynski, 1800 Barton Crek Blvd., Austin, TX, 78735-1606, US

(21) Appl. No.: 11/020,953

(22) Filed: Dec. 22, 2004

Related U.S. Application Data
(60) Provisional application No. 60/534,571, filed on Jan. 6, 2004.

(51) Int. Cl. A61B 5/04

(52) U.S. Cl. 600/513

(57) ABSTRACT

The invention relates to a method and device for simultaneous monitoring of cardiac activity, respiratory rate, and the evaluation of sleep states of a user. The invention comprises a seamless on-line evaluation of cardiac activity, heart rate variability, and ECG derived respiratory rate and using these data as an input for evaluation and quantitative assessment of sleep disorders. The hardware comprises of a flexible cardiac belt with 3 electrodes, which is easy to use without involvement of trained professionals.

### CROSS REFERENCE TO RELATED APPLICATIONS

This patent application is based upon Provisional Patent Application Ser. No. 60/534,571 filed on Jan. 6, 2004

### BACKGROUND OF THE INVENTION

The present invention relates to the field of sleep monitoring in particular to the simultaneous monitoring of cardiac functions, respiratory rates, and evaluation of sleep states of a user.

Sleeping disorders affect approximately 40 million Americans. According to the National Commission on Sleep Disorders Research the vast majority of patients with sleep disorders currently remain undiagnosed.

It is common that sleep disorders are associated with cardiovascular diseases or cause the development of cardiac abnormalities. Thus, simultaneous observation of the ECG and the respiratory cycle over long periods is often clinically useful.

Under normal circumstances an individual progresses through an orderly succession of sleep states and stages. The first cycle begins by going from wakefulness to Non-Rapid Eye Movement (NREM) sleep. NREM sleep is followed by Rapid Eye Movement (REM) sleep, and the two sleep states alternate throughout the night with an average period of about 90 minutes. A night of a normal human sleep usually consists of 4-6 NREM/REM sleep cycles.

To facilitate the diagnosis of sleep disorders, patients are monitored using polygraph recording of electroencephalograms (EEG), electrocardiogram (ECG), electro-oculogram (EOG) and other data.

Very often sleep evaluation is not possible without the use of sedative drugs because the plurality of electrodes connected to the patient inflicts anxiety and restrains the patient from a normal sleeping pattern. In these cases the validity of results are downgraded significantly.

The equipment used for sleep monitoring is costly and normally requires trained professionals for the operation and interpretation. Sleep monitoring usually is provided in specialized facilities (sleep labs) in a hospital environment.

The present invention enables true telemedicine sleep monitoring applications. This is not currently practical since the current state of the art is confined only to hospitals or acute care facilities.

### BRIEF DESCRIPTION OF THE DRAWINGS

FIG. 1 illustrates a one lead wearable cardiobelt and placement of electrodes.

FIG. 2 is a step-by-step diagram of ECG signals acquisition and analysis of heart activity and sleep stages.

FIG. 3 illustrates ECG waveform and characteristic points.

FIGS. 4a, 4b, 4c and 4d show detection and refining of point R.

FIG. 5 illustrates point and intervals of two successive RR intervals used in the calculation of noise level and point T.

FIGS. 6a and 6b illustrate saw-type and spike-type noise.

FIGS. 6c and 6d show ECG fragment before and after cubic spline smoothing.

FIGS. 7a, 7b and 7c illustrate detection of point Q.

FIGS. 8a, 8b and 8c illustrate detection of point S.

FIGS. 9a, 9b, 9c, 9d, 9e, 9f and 9g illustrate detection of points T and Tₑ.

FIG. 10 illustrates calculation of ECG parameters.

FIG. 11 shows ECG derived respiratory rate.

FIG. 12a-12c illustrates time-domain distribution of cardiac activity measured data.

FIG. 13a-13b demonstrates three main spectral components of the variability of the cardiac activity.

FIG. 14 illustrates steps of sleep evaluation.

FIG. 15 illustrates an example of the drift of the user from wakefulness stage to NREM sleep.

### DETAILED DESCRIPTION OF THE DRAWINGS

In the following description, numerous specific details are set forth to provide a thorough understanding of the present invention. However, it will be obvious to those skilled in the art that the present invention may be practiced without such specific details. For the most part, details concerning specific non-essential materials and the like have been omitted inasmuch as such details are not necessary to obtain a complete understanding of the present invention and are within the skills of persons of ordinary skill in the relevant art.

The fundamental aspect of the invention is a simultaneous monitoring of ECG and respiratory parameters, evaluation of autonomic nervous system (ANS) activity, and determining of sleep states incorporated in a wearable unobtrusive device which can be used for in-home, ambulatory and in-hospital sleep monitoring without supervision or assistance of trained personal.

These objectives are reached by using ECG signals for the measurement of ECG key parameters and ECG derived respiratory rate, evaluation of electrophysiological heart abnormalities, and evaluation of ANS's sympathetic and parasympathetic activity based on the heart rate variability.

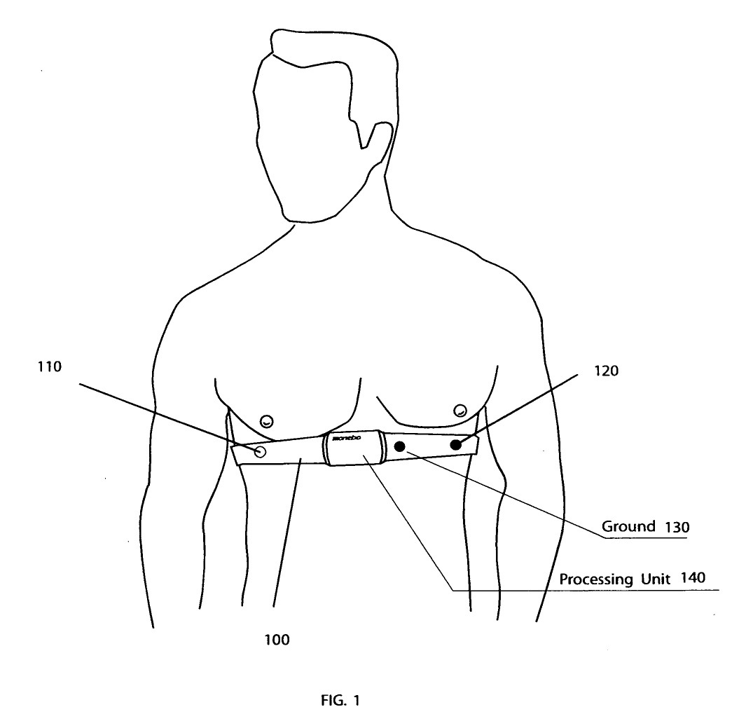

An embodiment of the invention is shown in FIG. 1 wherein a belt 100, with electrodes 110, 120, 130 and signal processing unit (“SPU”) 140 are shown. Cardiac signals are acquired through the electrodes 110, 120, 130 and are then conveyed over wiring embedded in the belt 100 to the SPU 140 where processing and transmission of the ECG signals take place.

The belt 100 is made, for example, from a flexible material that is light, soft, porous, non-slipping and comfortable to wear.

Three electrodes 110, 120, 130 are shown. One serves as a neutral electrode 130 while the other two electrodes 110, 120 are located at the modified bipolar lead I (MLI).

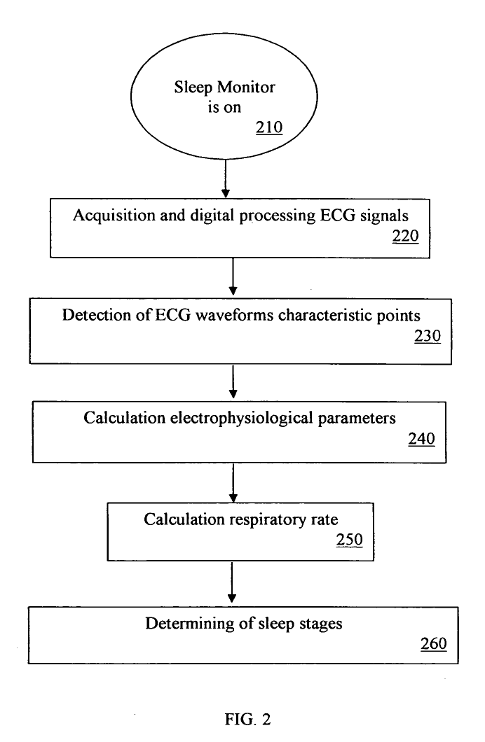

FIG. 2 illustrates steps of calculation of cardiac and respiratory parameters, assessment of heart electrophysiological abnormalities, and evaluation of sleep stages.

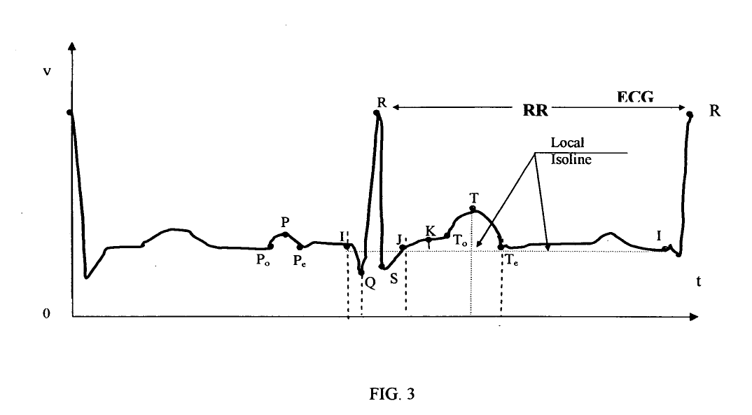

After activating the sleep monitor 210 and acquiring ECG 220 signals, the characteristic points (a.k.a. fiduciary points) Q, R, S, J, T and Tₑ of ECG (FIG. 3) are ascertained in step 230.

The detection of characteristic points Q, R, S, J, T and Tₑ and is illustrated in FIG. 3 to FIG. 9a-9e.

The detection of characteristic points starts from extraction of point R as the most distinctive point of ECG.

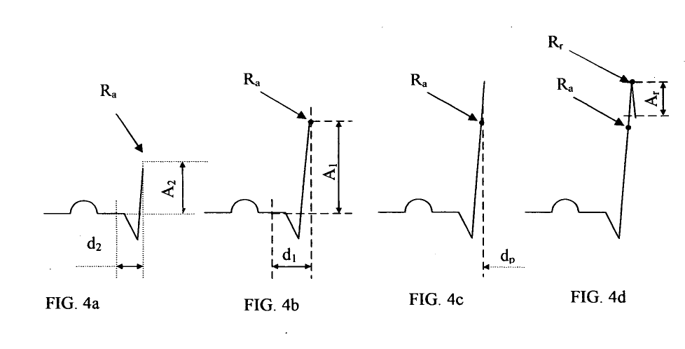

When progressing along axis t (FIG. 3) and comparing amplitude, Vᵢ, of a point at current time, tᵢ, and amplitudes, V₁ at time tᵢ−d₁ and V₂ at time tᵢ−d₂ (FIG. 4a-4d), the approximate location of point Rₐ is found, when the following is true:
(Vᵢ−V)>A₁ OR (Vᵢ−V₂)>A₂
Where:
Vᵢ=amplitude of the current point at time tᵢ;
V₁=amplitude at time tᵢ−d₁;
V₂=amplitude at time tᵢ−d₂;

A₁=0.25 mV and d₁=75 ms may be used for strongly expressed (high) R waves (5b).

A₂=0.15 mV and d₂=40 ms may be applied for weakly expressed (short) R waves (5a).

Theses values are commonly selected by those of skill in the art because the amplitude of point R normally increases 0.25 mV within a period of 75 ms for high R waves and it increases 0.15 mV within a period of 40 ms for short R waves. However, persons of ordinary skill in the art realize certain physiological conditions may require the alteration of values A₁, A₂, d₁ and d₂.

After a point Rₐ is found, the location Rᵣ of point R is ascertained analyzing a time interval [tᵢ, tᵢ+dᵣ] (FIG. 4d). As soon as the amplitude of point R is decreases by more than Aᵣ, the location of point R is considered found and further analysis of interval [tᵢ, tᵢ+dᵣ] is stopped. The point Rᵣ is one step back from the point of decrease. The empiric values of dᵣ and Aᵣ are 200 ms and 0.05 mV respectively, because the maximum width of R wave is not more than 200 ms and, within this period, R wave descends by not less than 0.05 mV. However, persons of ordinary skill in the art may successfully use other values.

RR interval is the time between two successive R points (FIG. 5). QRS fragment is defined as two successive RR intervals (RR)ᵢ₋₁ and (RR)ᵢ (FIG. 5). Each current (RR)ᵢ interval is tested for the level of noise.

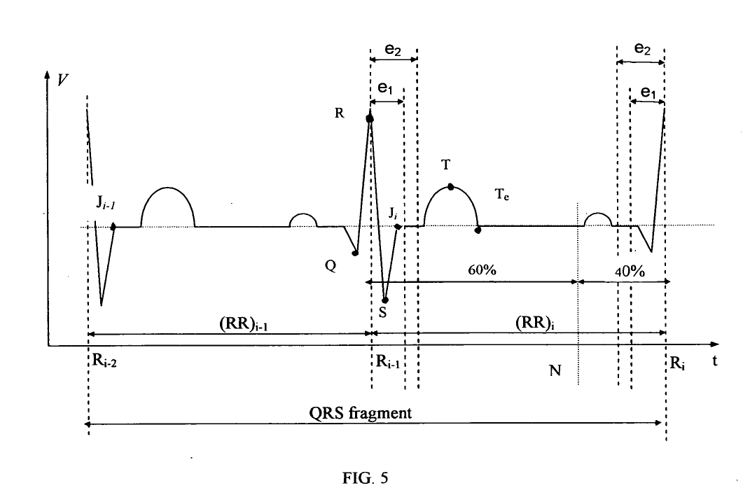

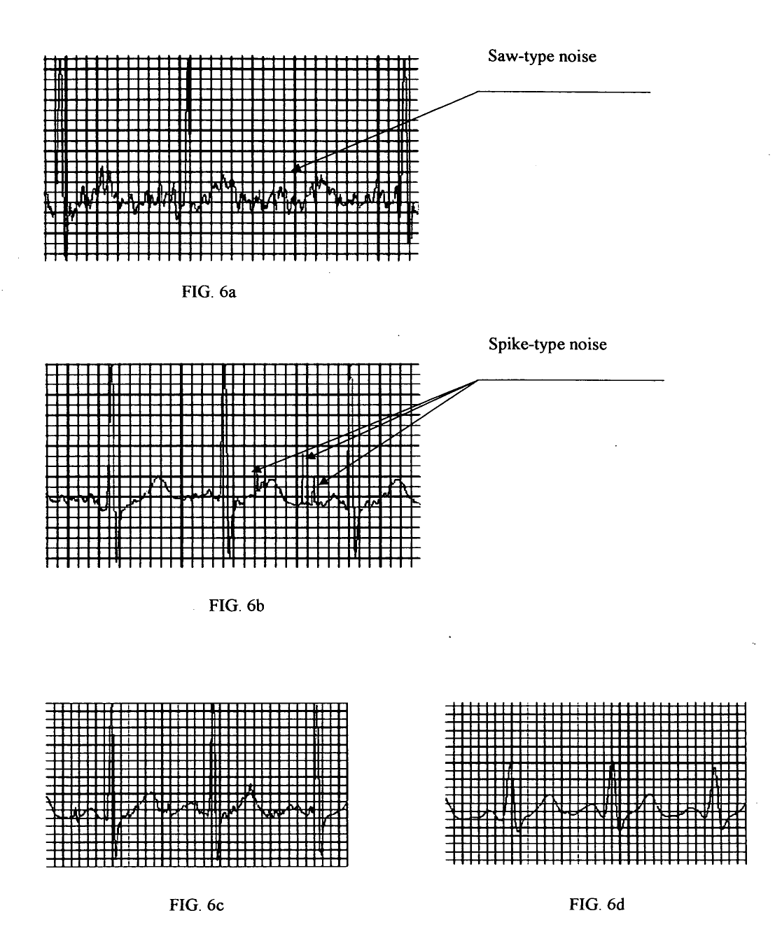

First the noise level, N₁, of saw-type noise (FIG. 6a) is calculated within time interval [Rᵢ₋₁+e₁, Rᵢ−e₁], (FIG. 5), where e₁ may typically be 75 ms.

Starting N₁=0, for each point j of interval [Rᵢ₋₁+e₁, Rᵢ−e₁] and each m, if |Vⱼ−Vⱼ₋₁|>2^{m} AND |Vⱼ−Vⱼ₊₁|>2^{m}, then N₁=N₁+2^{m}, where m=3, 2, 1, 0.

At the next step the level N₂ of spike-type noise (FIG. 6b) is calculated within time interval [Rᵢ₋₁+e₁, Rᵢ−e₁], (FIG. 5b), where e₁ may typically be 115 ms.

Starting N₂=0, for each point j of interval [Rᵢ₋₁+e₁, Rᵢ−e₁] and each m, if |Vⱼ−Vⱼ₋₁|>m AND |Vⱼ−Vⱼ₊₁|>m, then N₁=N₁+m, where m=30, 20.

The total noise level of current interval (RR)ᵢ, Nᵢ=N₁+N₂. If the noise level Nᵢ>Nₗᵢₘᵢₜ where Nₗᵢₘᵢₜ may be 20, then current interval (RR)ᵢ is considered unreliable and excluded from further calculations.

The values e₁=75 ms and e₂=115 ms are empirically derived and commonly selected by those of skill in the art because indentations 75 ms and 115 ms from R point exclude Q, S and T waves from mistakenly considering these points as saw-type noise as well as point R as a spike-type noise. However, persons of ordinary skill in the art may successfully use other values.

Nₗᵢₘᵢₜ=20 provides a sufficient noise filtering for disclosed application however, persons of ordinary skill in the art may successfully use other values.

After noise filtering of RR interval, RR interval is smoothed using cubic spline interpolation algorithm included in Matlab Version 3.2 spline toolbox. However, persons of ordinary skill in the art may successfully use other smoothing techniques. FIG. 6c illustrates ECG fragment before smoothing, while FIG. 6d shows ECG fragment after cubic spline smoothing was applied.

QRS fragment (FIG. 5) is defined as two successive reliable RR intervals, (RR)ᵢ₋₁ and (RR)ᵢ.

Point Q, S, J, T, and Tₑ are ascertained within QRS fragment.

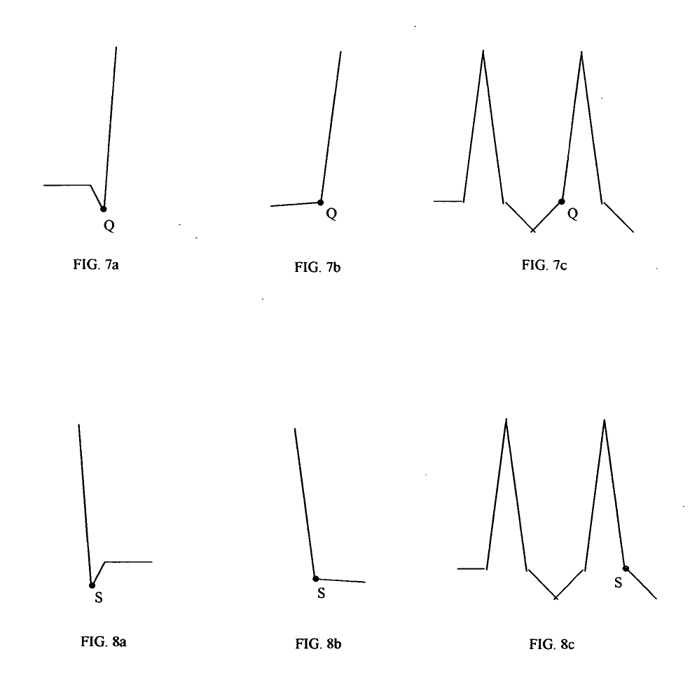

Point Q is calculated from the graphs in FIGS. 7a-7c in the following manner:

Referring to FIG. 7a and recalling that R has already been located as shown above, point Q may be obtained as follows:

A time interval to be analyzed is defined as [t_{DQ}, t_{R}] where, typically, t_{DQ}=t_{R}−75 ms. t_{R} corresponds with point R as was derived above. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:
Aᵢ₋₁>Aᵢ and (A_{R}−Aᵢ)>A_{RQ}
Where:
A denotes amplitude
A_{RQ}=0.1 mV
A_{R}=the amplitude at point R

A_{RQ}=0.1 mV is commonly selected by those of skill in the art because a typical R peak is at least 0.1 mV “above” the Q. However, persons of ordinary skill in the art may select other value, which may be successfully applied. This approach is valid for normal Q waves as shown on FIG. 7a.

Next, if the above conditions are not met and Q is not ascertained, the following conditions are evaluated to determine Q (See FIG. 7b):

A time interval to be analyzed is defined as [t_{DQ,} t_{R}] where, again, typically, t_{DQ}=t_{R}−75 ms. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:
(Aᵢ−Aᵢ₋₃)>A_{d} and (A_{R}−Aᵢ)>A_{RQ}
Where:
A denotes amplitude
A_{d}=0.025 mV
A_{RQ}=0.1 mV

A_{RQ}=0.1 mV is commonly selected by those of skill in the art because a typical R peak is at least 0.1 mV “above” the Q. However, persons of ordinary skill in the art may select other value, which may be successfully applied.

A_{d}=0.025 mV is commonly selected by those of skill in the art because this amplitude difference is typical for abnormal Q wave shown on FIG. 7b. However, persons of ordinary skill in the art may select other value, which may be successfully applied.

Next, if the above conditions are not met and Q is not ascertained, the following conditions are evaluated to determine Q (See FIG. 7c):

A time interval to be analyzed is defined as [t_{DQ,} t_{R}] where, again, typically, t_{DQ}=t_{R}−75 ms. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:

$$\frac{{A}_{i} - {A}_{i - 3}}{{A}_{i + 3} - {A}_{i}} \geq {Q}_{r} and ( {A}_{R} - {A}_{i} ) > {A}_{RQ}$$

Where:
A denotes amplitude
A_{RQ}=0.1 mV
Qᵣ=0.45

A_{RQ}=0.1 mV is commonly selected by those of skill in the art because a typical R peak is at least 0.1 mV “above” the Q. However, persons of ordinary skill in the art may select other value, which may be successfully applied.

Qᵣ=0.45 is commonly selected by those of skill in the art because it's a typical ratio for abnormal Q wave related to group of premature beats (FIG. 7c). However, persons of ordinary skill in the art may select other value, which may be successfully applied.

Referring to FIG. 8a and recalling that R has already been located as shown above, point S may be obtained as follows:

A time interval to be analyzed is defined as [t_{R}, t_{DS}] where, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:
Aᵢ₊₁>Aᵢ and (A_{R}−Aᵢ)>A_{RS}
Where:
A denotes amplitude
A_{RS}=0.1 mV

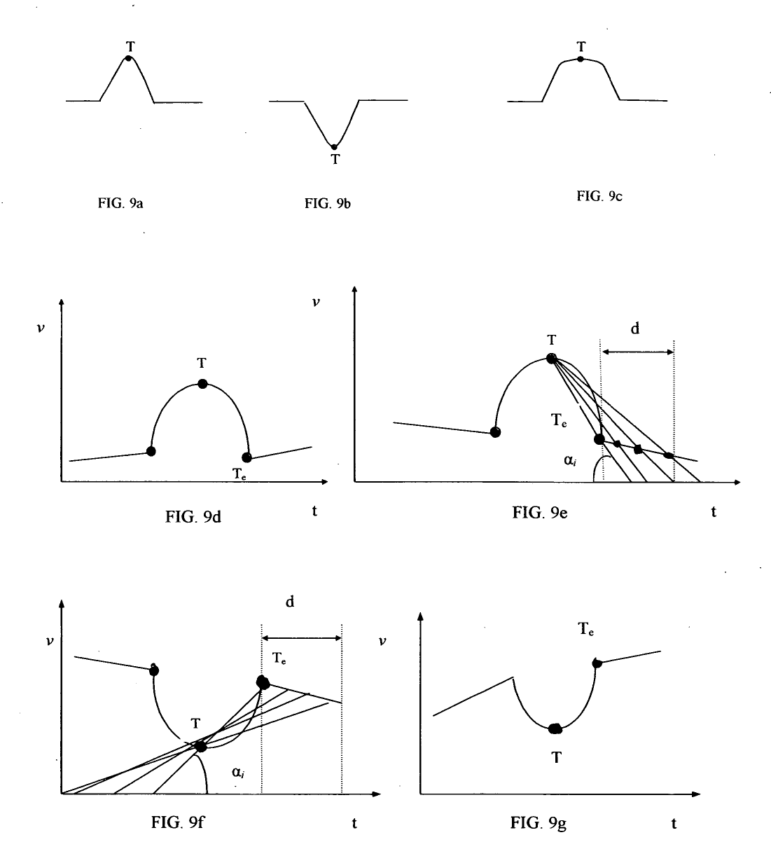

A_{RS}=0.1 mV is commonly selected by those of skill in the art because a typical R peak is at least 0.1 mV “above” the S. However, persons of ordinary skill in the art may select other value, which may be successfully applied. This approach is valid for normal S waves as shown on FIG. 9a.

Next, if the above conditions are not met and S is not ascertained, the following conditions are evaluated to determine S (See FIG. 8b):

A time interval to be analyzed is defined as [t_{R}, t_{DS}] where, again, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:
(Aᵢ−A₁₊₃)<A_{d} and (A_{R}−Aᵢ)>A_{RS}
Where:
A denotes amplitude
A_{d}=0.025 mV
A_{RS}=0.1 mV

A_{RS}=0.1 mV is commonly selected by those of skill in the art because a typical R peak is at least 0.1 mV “above” the S. However, persons of ordinary skill in the art may select other value, which may be successfully applied.

A_{d}=0.025 mV is commonly selected by those of skill in the art because a this amplitude difference is typical for abnormal S wave shown on FIG. 8b. However, persons of ordinary skill in the art may select other value, which may be successfully applied.

Next, if the above conditions are not met and S is not ascertained, the following conditions are evaluated to determine S (See FIG. 8c):

A time interval to be analyzed is defined as [t_{R}, t_{DS}] where, again, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:

$$\frac{{A}_{i} - {A}_{i + 3}}{{A}_{i - 3} - {A}_{i}} \geq {S}_{r} and ( {A}_{R} - {A}_{i} ) > {A}_{RS}$$

Where:
A denotes amplitude
A_{RS}=0.1 mV
Sᵣ=0.3

A_{RS}=0.1 mV is commonly selected by those of skill in the art because a typical R peak is at least 0.1 mV “above” the S. However, persons of ordinary skill in the art may select other value, which may be successfully applied.

Sᵣ=0.3 is commonly selected by those of skill in the art because it's a typical ratio for abnormal S wave related to group of premature beats (FIG. 8c). However, persons of ordinary skill in the art may select other value, which may be successfully applied.

Recalling that point S has already been located as shown above, point J (FIG. 3) may be obtained as follows:

A time interval to be analyzed is defined as [t_{S}, t_{DJ}] where, typically, t_{DJ}=t_{S}+75 ms. A 75 ms period is commonly selected by those of skill in the art because the point J is normally within 0 to 75 ms of S. However, persons of ordinary skill in the art may select other value, which may be successfully applied.

When sampling this period, starting at t_{R} and progressing towards t_{DJ}, J is found when the following is true:
(Aᵢ₊₃−Aᵢ)<A_{d}
Where:
A denotes amplitude
A_{d}=0.025 mV

If point J is not ascertained, point J may be considered to have time coordinate equal t_{Q}+50 ms.

Recalling that point J (FIG. 3) has been located and two successive RR intervals, (RR)ᵢ₋₁ and (RR)ᵢ (FIG. 5) have been identified as shown above, point T (FIG. 3, FIGS. 9a-9e) may be obtained as follows:

A time interval to be analyzed is defined as [t_{J}, t_{N}], where t_{N}=60%*[Rᵢ₋₁, Rᵢ] (FIG. 6). 60% is commonly selected by those of skill in the art, because the point T is normally located within interval [t_{J}, t_{N}]. However, persons of ordinary skill in the art may select other value, which may be successfully applied.

When sampling this period, starting at t_{J} and progressing towards t_{N}, point T is found if the distance from moving point (tᵢ,Aᵢ) to straight line (Jᵢ,Jᵢ₋₁), which exists between point Jᵢ of interval [Rᵢ₋₂, Rᵢ] and point Jᵢ₋₁ of interval [Rᵢ₋₁, Rᵢ₋₁] (FIG. 5), is more than T_{Amax}. Where T_{Amax} is a maximum point and =2 mm. Those of skill of the art commonly select this value, however, persons of ordinary skill in the art may select other value, which may be successfully applied. This approach is valid for normal T wave as it is shown on FIG. 9a.

If T wave is inverted (FIG. 9b), then, when sampling this period, starting at t_{J} and progressing towards t_{N}, point T is found if the distance from moving point (tᵢ, Aᵢ) to straight line, (Jᵢ,Jᵢ₋₁), which exists between point Jᵢ of interval [Rᵢ₋₂, Rᵢ] and point Jᵢ₋₁ of interval [Rᵢ₋₁, Rᵢ₋₁] (FIG. 5), is more than T_{Amin}. Where T_{Amin} is a minimum point and =8 mm. This value is commonly selected by those of skill of the art, however, persons of ordinary skill in the art may select other value, which may be successfully applied. This approach is valid for a normal T wave as it is shown on FIG. 9b.

For flat T waves (FIG. 9c), sampling interval [tⱼ, t_{N}] as defined above and progressing toward t_{N}, point T is found when the following is true:
(Aᵢ−Aᵢ₊₅)>A_{d}
Where:
A denotes amplitude
A_{d}=0.025 mV

A_{d}=0.025 mV is commonly selected by those of skill in the art because experimental data well correlated with this value. However, persons of ordinary skill in the art may select other value, which may be successfully applied.

If point T is not identified, the point is considered undetectable. Time coordinates of point T and point Tₑ are then considered equal to t_{N}.

When point T has been located, then point Tₑ (FIGS. 9d, 9e) is ascertained as follows:

A time interval to be analyzed starting from t_{T} and progressing to direction of time gain.

Point Tₑ is found when one of the following is true:
Aᵢ>Aᵢ₋₁, if T wave has shape as shown on FIG. 9d
   or
Aᵢ<Aᵢ₋₁, if T wave has shape as shown on FIG. 9f
   or
angle αᵢ, between straight line (T, Tᵢ) and axis t, becomes less than αᵢ₋₁ and this condition remains for the period of time not less then d=40 ms for T waves shaped as shown on FIG. 9e and FIG. 9g.

d=40 ms is commonly selected by those of skill in the art because experimental data well correlates with this value. However, persons of ordinary skill in the art may select other value, which may be successfully applied.

Other methods such as spectral analysis, signal averaging, wavelet transform, and Fourier transform may be successfully used for detection of characteristic points of ECG.

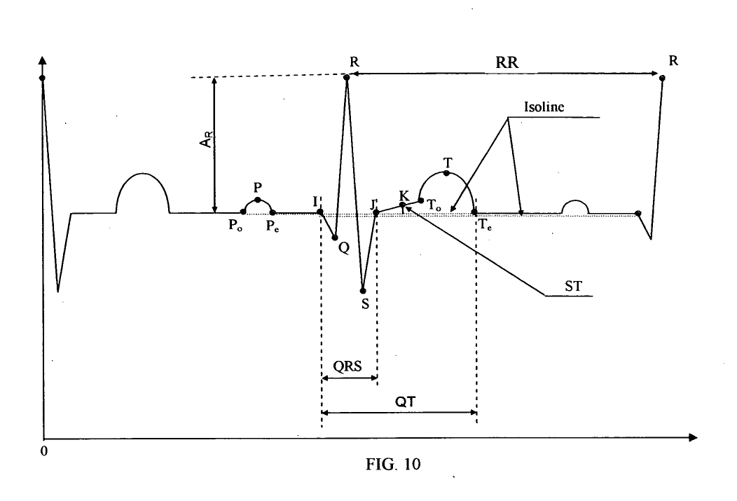

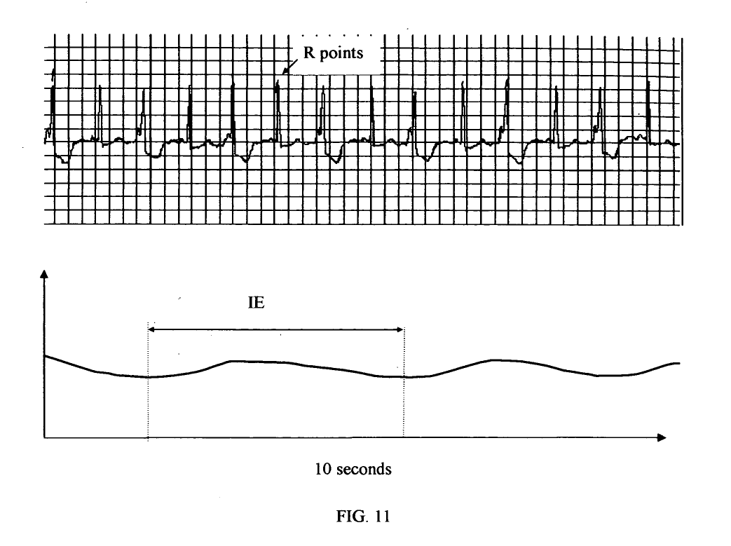

In step 240 electrophysiological parameters RR interval, width of QRS, QT interval, T wave inversion, and ST deviation are calculated using amplitudes and time values of ascertained characteristic points (FIG. 10). Calculated parameters are compared with threshold values. If a parameter is out of threshold range, the parameter is marked as cardiac events, which significance is defined using Green-Yellow-Red concept.

Recalling that the electrophysiological parameters have been calculated, heart rate (HR), premature beats, bundle branch blocks, and bradycardia and tachycardia arrhythmias may be detected.

Referring to QRS widths and RR intervals, all beats may be classified as: normal beats, supraventricular premature beats, ventricular premature beats, and unclassified beats. During respiration amplitudes A_{R} of points R (FIG. 10) are influenced by motion of the electrodes with respect to the heart, and by changes in the electrical impedance of the thoracic cavity. These physical influences of respiration result in amplitude variations, which are used for calculation of respiratory rate 250. FIG. 11 shows variation of R wave amplitude (upper waveform) and respiratory trace (lower waveform), which is calculated using cubic spline interpolation of peaks of R waves.

Respiratory/breathing rate (BR) is calculated for each breathing cycle as:

$$BR = \frac{60}{IE}$$

Where:
BR=respiratory rate in cycles per min;
IE=length of breathing cycle (FIG. 11).

Artifacts and premature beats may affect the calculation of respiratory rate. While unreliable and noisy intervals have been excluded from observation (FIG. 6a-6d), respiratory cycles, which include unclassified beats, are ignored and excluded from further calculation.

In step 260, heart rate variability (HRV) is examined using power spectral analysis method.

Heart rate is not the same for each cardiac cycle (beat). The heart rate varies from beat to beat. This fluctuation is controlled by sympathetic and parasympathetic branches of the autonomic nervous system (ANS) and reflects the individual's capacity to adapt effectively to environmental demands. The parasympathetic and sympathetic divisions of the ANS constantly cooperate, either facilitating or inhibiting cardiovascular functions. There is a direct correlation between variability of heart rate and activity of parasympathetic and sympathetic systems.

During the transition from wakefulness to sleep and going through sleep stages, dramatic changes occur in the functions sympathetic and parasympathetic systems. It has been shown in many studies, that during the transition from wakefulness to sleep, sympathetic activity decreases from 53±9% of total power to 41±5%, while parasympathetic activity markedly increases from 19±4% to 40±6%. During REM sleep sympathetic activity doesn't change, while parasympathetic activity decreases by 17±2%.

One of the objectives of the present invention is providing quantitative assessment of sympathetic and parasympathetic activity and their overtime changes by analyzing heart rate variability. This data is used to discriminate wakefulness state, and sleep stages of the user.

Heart rate (HR) is defined as:

$$HR = \frac{60}{RR} 1000 ;$$

Where:
HR=heart rate;
RR=RR interval in ms (FIG. 10)

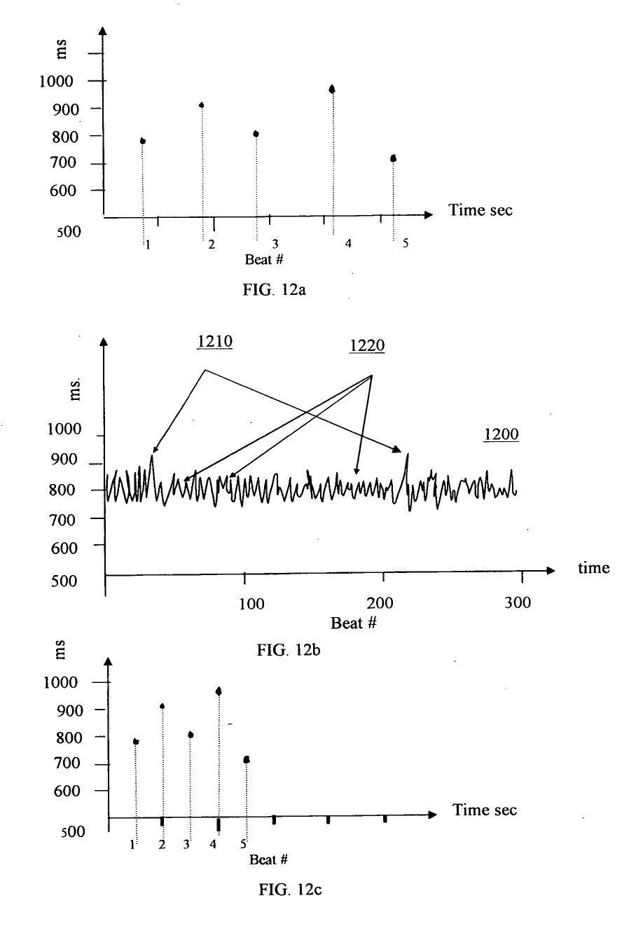

FIG. 12a shows an example of distribution plot of measured RR interval of 5 successive normal cardiac cycles (beats). Due to the time fluctuation between cardiac cycles, the plot represents irregularly time-sampling signals.

FIG. 12b shows an irregular sampled plot (tachogram) 1200 of 300 successive normal RR, values. The ordinate represents measured values of RRᵢ. Spikes 1210 are due to increased sympathetic activity, while low fluctuations area 1220 points on prevalence of parasympathetic activity.

Traditional spectral analysis methods are not able to process irregular sampled signals. In the present invention irregular sampled tachogram is resampled using sampling rate equal half of the average interval found in the time-domain tachogram. This rate is compliant with Nyquist theorem (i.e. sampling rate is higher than twice the highest frequency contained in the signal) and at the same time it is low enough for effective usage of processing power. However, persons of ordinary skill in the art may successfully use other values.

FIG. 12c shows an example of resampling of the tachogram shown on FIG. 12a. Resampled points are repositioned at the new sampling interval equal to the half average interval found in the tachogram shown on FIG. 12a.

The resampled tachogram is used as an input for the Fourier Transform spectral analysis.

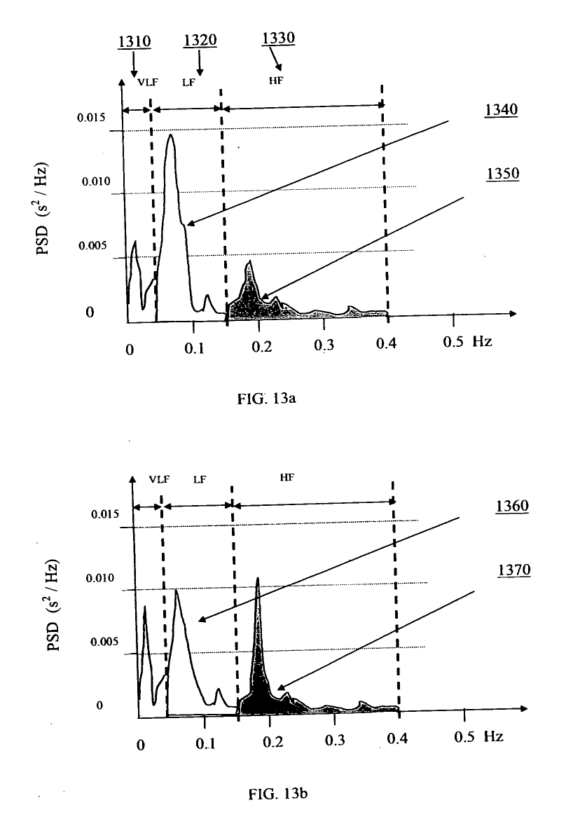

The total power spectrum of the resampled tachogram is divided into three main frequencies, FIG. 13a-13b:
the very low frequency range (VLF) 0.0033 to 0.04 Hz, discriminates slower changes in HRV and reflects sympathetic activity 1310;
low frequency range (LF) 0.04 to 0.15 Hz representing both sympathetic and parasympathetic activity 1320;
and high frequency (HF) 0.15 to 0.4 discriminates quicker changes in the HRV and reflecting parasympathetic activity 1330.

The power spectrum division on 0.0033 to 0.04, 0.04 to 0.15, and 0.15 to 0.4 is defined by a standard developed by Task Force of European Society of Cardiology and North American Society of Pacing and Electrophysiology, European Heart Journal (1966), 17, 354-381.

In the present invention, Fast Fourier Transform (FFT) algorithm is applied to evaluate the discrete-time Fourier transform (DFT) of N equispaced samples of a 5 minutes time-series of records of HRV. Five minutes time-series is recommended by the standard developed by Task Force of European Society of Cardiology and North American Society of Pacing and Electrophysiology, European Heart Journal (1966), 17, 354-38. However, persons of ordinary skill in the art may successfully use other values.

The number of operations required to calculate the FFT is proportional to log₂N when N is an integer power of 2. In the present invention N=1024 and covers the maximum possible number of samples of 5 minutes of the time series. The number of samples is artificially increased by adding zero-value samples (zero-padding), if the number of samples is less than 1024.

Standard, off-the-shelf FFT software is used for calculation of the total spectrum power (TP), power spectrum distribution, and calculation of sympathetic and parasympathetic spectral powers, e.g. FFTW Version 3.0.1, Matlab, The Mathworks. However, persons of ordinary skill in the art may successfully use other FFT software.

FIG. 13a illustrates an example of calculated distribution of power spectrum density (PSD) for a person in wakefulness state 1340 and 1350.

FIG. 13b shows an example of calculated power spectrum distribution 1360 and 1370 when a person is in the NREM sleep state. The transition from wakefulness to NREM sleep is marked by a progressive decrease in LF spectrum power reflecting sympathetic activity and a significant increase in HF spectrum power reflecting parasympathetic activity.

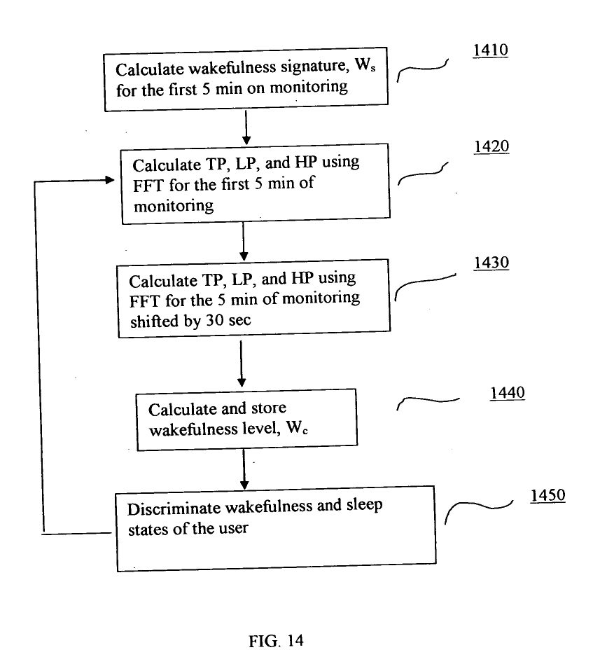

FIG. 14 illustrates steps of evaluation and decimation of wakefulness and transition to the sleep state of the user.

The first step 1410, total power (TP), LF activity power (LP), and HF activity power (HP) are calculated for the first 5 minutes of monitoring using FFT algorithm.

The second step 1420, wakefulness signature level WS is calculated as ratio of LP to HP.

$${W}_{s} = \frac{{LP}_{s}}{{HP}_{s}}$$

Where:
Wₛ=wakefulness signature value,
LPₛ=signature sympathetic activity power,
HPₛ=signature parasympathetic activity power.

Next step, 5 minutes time interval is shifted by 30 seconds and the new set of TP_{c}, LP_{c} and HP_{c} is calculated 1430.

In step 1440 the current wakefulness level W_{c} is calculated:

$${W}_{c} = \frac{{LP}_{c}}{{HP}_{c}}$$

The calculated value W_{c} is stored in FIFO buffer. The buffer contains data of 5 minutes of monitoring.

The transition from wakefulness to NREM sleep is characterized by the rapid shift of predominance of sympathetic activity to predominance of parasympathetic activity.

Sympathetic power, LP drops up to 10%, while parasympathetic power, HP increases up to 50%. These changes occur during time interval from 1 to 5 minutes.

In step 1450 the current wakefulness level W_{c} is compared with wakefulness signature level Wₛ.

If W_{c}≦0.75*Wₛ, (i.e. ratio of sympathetic activity to parasympathetic activity drops by equal or more then 25% during the last 5 minutes), then the user is considered in NREM sleep state. Twenty five percent drop is cited by all studies however, persons of ordinary skill in the art may successfully use other percent decrease.

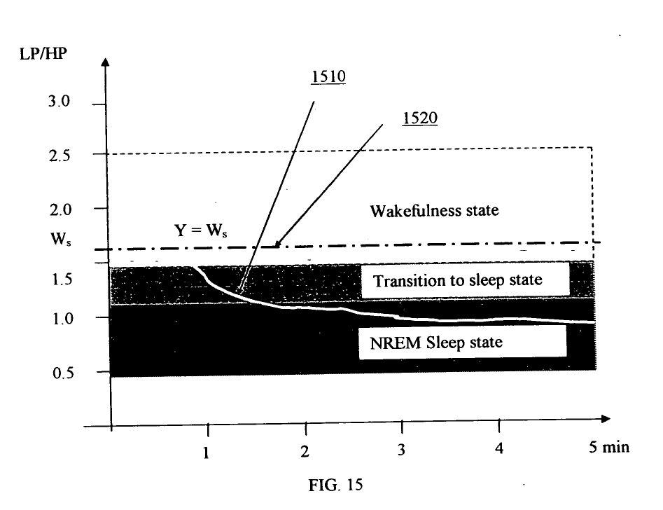

An example of the drift 1510 of the user from wakefulness to sleep state is shown in FIG. 15. The ratio LP/HP dropped by more than 25% within less then 4 minutes. The line 1520 shows the baseline of the wakefulness signature of the user.

If W_{c} increases by more than 15% and LP doesn't change and the previous record shows that the user was in NREM stage, then the user is considered in REM sleep.

If W_{c} increases by more than 15% and LP increases and the previous record shows that the user was in NREM or REM sleep, then the user is considered in the after-sleep wakefulness state.

1. A method of assessing the cardiac, respiratory and sleep activity of a user comprising:
a) Placing a plurality of electrodes on said user's torso;
b) Acquiring an ECG using at least one of said electrodes;
c) Identifying one or more characteristic points on said ECG;
d) Determining respiratory rate using R-waves of said acquired ECG;
e) Measuring parameters using at least one of said one or more characteristic points on said ECG; and
f) Measuring heart rate variability using said measured parameters of said ECG;
g) Evaluation of sympathetic and parasympathetic activity of autonomic nervous system using said measured heart rate variability;
h) Assessment of sleep stages of a user using power spectrum analysis of said sympathetic and parasympathetic activity.

2. The method of claim 1, wherein said plurality of electrodes comprise two sensing electrodes and one reference electrode positioned substantially parallel to the subclavian artery where sensing electrode are positioned in a close proximity to midaxillary lines at nipples level.

3. The method of claim 2, wherein said sensing electrodes are placed in a stretchable strap.

4. The method of claim 1, wherein said step of identifying one or more characteristic points on said ECG entails identifying at least one of the following points: R, Q, S, J, T and Tₑ.

5. The method of claim 1, wherein said characteristic points are used for measurement of cardiac parameters.

6. The method of claim 1, wherein said respiratory rate is measured using fluctuation of the amplitude of R wave of said ECG.

7. The method of claim 1, wherein power spectrum analysis is used for determining of said sleep stages.

8. The method of claim 1, wherein Fast Fourier Transform (FFT) algorithm applied for calculation of low frequency (LF), reflecting both sympathetic and parasympathetic activities, and high frequency (HF), reflecting parasympathetic activity.

9. The method of claim 8, wherein total power (TP), said LF activity power (LP), and said HF activity power are calculated for the first 5 minutes of monitoring using said FFT algorithm.

10. The method of claim 8, wherein the wakefulness signature level WS is calculated by:
$${W}_{s} = \frac{{LP}_{s}}{{HP}_{s}}$$
Where:
Wₛ=wakefulness signature value,
LPₛ=signature sympathetic activity power,
HPₛ=signature parasympathetic activity power.

11. The method of claim 8, wherein current said wakefulness level W_{c} is calculated in 5 minutes window with 30 second shift by:
$${W}_{c} = \frac{{LP}_{c}}{{HP}_{c}}$$
Where:
W_{c}=current wakefulness value,
LP_{c}=current sympathetic activity power,
HP_{c}=current parasympathetic activity power.

12. The method of claim 8, wherein in order to determine said sleep stages said current wakefulness level is compared with said wakefulness signature level.

13. The method of claim 8, wherein if said W_{c}≦0.75*Wₛ, (i.e. ratio of sympathetic activity to parasympathetic activity drops by equal or more then 25% during the last 5 minutes), then the user is considered in NREM sleep state.

14. The method of claim 8, wherein if said W_{c} increases by more than 15% and said LP doesn't change and the previous record shows that the user was in NREM stage, then the user is considered in REM sleep state.

15. The method of claim 8, wherein if said W_{c} increases by more than 15% and LP increases and the previous record shows that the user was in NREM or REM sleep, then the user is considered in the after-sleep wakefulness state.
